(19) United States
(12) Patent Application Publication
(10) Pub. No.: US 2019/0328916 A1
(43) Pub. Date: Oct. 31, 2019

(54) METHOD AND APPARATUS FOR TRANSMITTING MICROBES DESTROYING UV LIGHT FROM A LIGHT SOURCE TO A TARGET

(71) Applicants: Risto KOPONEN, Rautalampi (FI); Petri JOKIMÄKI, Varkaus (FI)

(72) Inventors: Risto KOPONEN, Rautalampi (FI); Petri JOKIMÄKI, Varkaus (FI)

(21) Appl. No.: 16/465,104

(22) PCT Filed: Nov. 22, 2017
(86) PCT No.: PCT/FI2017/050811

§ 371 Date: May 29, 2019

(30) Foreign Application Priority Data
Nov. 30, 2016 (FI) 20165913

(51) Int. Cl. A61L 2/10; A61L 2/00

(52) U.S. Cl. CPC A61L 2/10; A61N 5/0624; A61L 2/0047

(57) ABSTRACT

The object of the invention is a method and an apparatus for transmitting microbe-destroying UV light produced with one or more UV LEDs from a light source to the target of the light. In the solution according to the invention, the properties of the UV light being produced are adjusted according to the need of the target, and the UV light is transmitted from the light source to the target of the light via a flexible optical fiber element. In addition to transmitting microbe-destroying UV light, other functions enabled by optical fibers are transmitted via the same optical fiber element.

The object of the present invention is a method as presented in the preamble of claim 1 and an apparatus as presented in the preamble of claim 11 for transmitting microbe-destroying UV light from a light source to a target.

The method and apparatus according to the present invention, hereinafter more briefly the solution, is suited for use advantageously in many different sites requiring the destruction of microbes, such as e.g. the healing of wounds and inflammations, the destruction of cancer cells, the disinfection of fluid administered intravenously in connection with a dripfeed, i.e. infusion, the disinfection of donor blood in conjunction with blood transfusion and the disinfection of, inter alia, foodstuffs to be fed in a fluid form into sales containers and storage containers in connection with putting food into receptacles.

Disinfection is generally defined as a method whereby microbes are destroyed by an inanimate material with chemical methods for preventing various infections. Disinfection targets are e.g. various surfaces, such as door handles, water taps in public premises and other touchable objects, machines and devices, medical equipment and also household water. When microbes in the tissues or on the skin of a person or animal are destroyed, the process is generally called antisepsis instead of disinfection. In the context of this description, the common designation ‘destruction of microbes’ refers to both disinfection and antiseptic treatment, but the terms ‘disinfection’ and ‘treatment’ are also used to refer to the same concepts.

In solutions known in the art the destruction of microbes is implemented with, inter alia, heat treatment, chemicals and also with light from UV fluorescent tubes. One known heat treatment method is pasteurization wherein, e.g. when treating milk, the milk is rapidly heated to a certain temperature and then cooled back to e.g. its storage temperature. A problem in pasteurization and in other heat treatment methods is the high energy consumption and also the fact that heat treatment alters the molecular structure of the liquid being treated. Yet another problem is that pasteurization produces greenhouse gases. A further problem is that pasteurization is not suited to e.g. treating and healing wounds nor to the disinfection of a fluid.

Correspondingly, in the destruction of microbes implemented with chemicals, residues of the chemicals used such as e.g. chlorine or ozone, often remain on the object or in the substance to be cleaned. These residues might be harmful to health. In addition, products of reaction from the chemicals used might remain on the object or in the substance being cleaned, which products change the properties of the object or substance being cleaned. Chemical methods thus alter the molecular structure of the substance, such as a liquid, to be treated. Chemical methods are indeed suited to the cleaning and disinfection of wounds, but not to healing them.

One solution that has been used for the destruction of microbes is UV light, which at certain wavelengths has properties destroying microbes or their DNA. UV fluorescent tubes have mainly been used as a light source in such cases, and the advantage of them is that they do not change the properties of the object or substance to be cleaned. A problem in these, however, is that fluorescent tube lamps are imprecise in their disinfection properties, in which case use of them cannot be targeted at destroying exactly certain microbes. This imprecision is increased by the fact that the properties of fluorescent tubes change as they age. Moreover, fluorescent tubes consume a lot of energy, are easily breakable and have a relatively short service life, and also when their service life expires they are hazardous waste because they contain, inter alia, mercury. Generally, it has not been possible to disinfect other than a clear liquid, i.e. in practice water, as well as air or solid materials with fluorescent tubes.

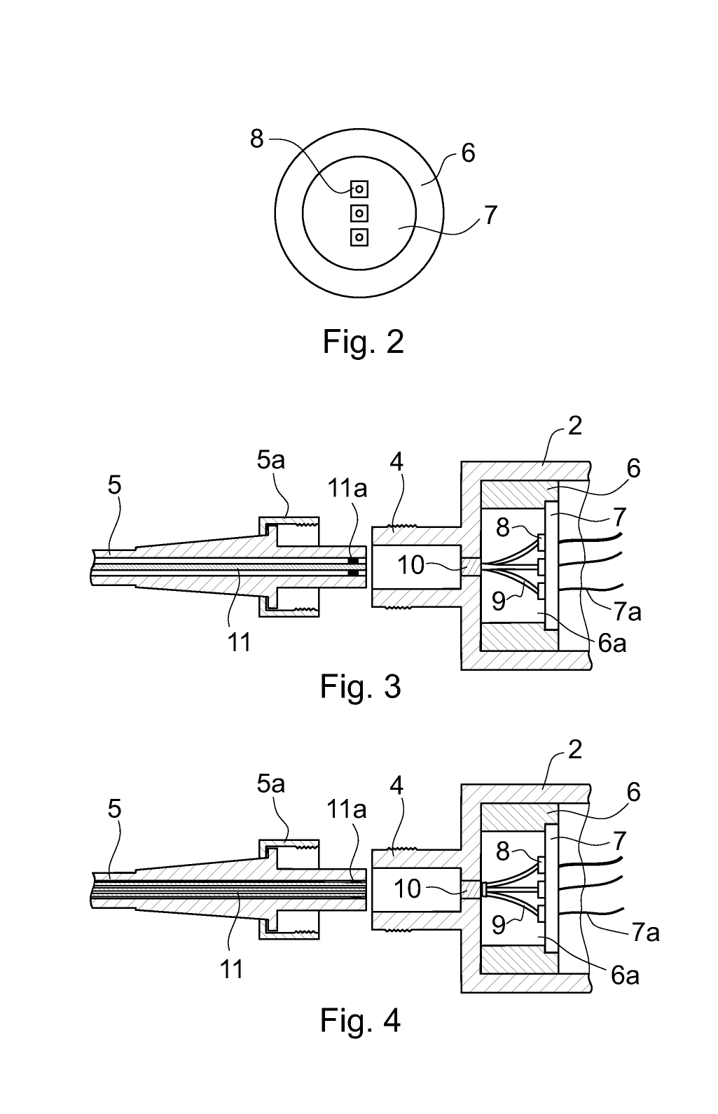

US patent publication US20150297767 A1 presents a sterilization solution wherein ultraviolet light produced with ultraviolet LEDs is directed to the sterilization target via a flexible optical fiber element, which optical fiber element can comprise fused silica fibers. The specification refers to sensors that measure the ultraviolet radiation directed to the object to be sterilized. Likewise, the specification states that the solution comprises a computer with which adjustment of the ultraviolet radiation dose is performed on the basis of the measurement results. Adjustment of the radiation dose in this case occurs automatically by means of the computer. There is no mention of manual adjustment. Furthermore, there is no mention in the publication of using an optical fiber element intended for directing UV light also to illuminate the object to be sterilized and to photograph the object to be sterilized with a camera via the optic fiber. The situation according to FIG. 2 of the US publication is extremely awkward for a patient and can even be painful, because the user of the sterilization device cannot see where the sterilization head of the optical fiber element is nor where it is going. In such a case, also the sterilization result is not the best possible. In addition, the solution according to the US specification, together with the computer, is expensive and complex.

A drawback common to all the aforementioned solutions, known in the art, for the destruction of microbes is further that they are extensive in their effect, so they cannot perform a precisely targeted process for destroying microbes.

The aim of the present invention is to eliminate the aforementioned drawbacks and to provide an environmentally friendly, energy-efficient, inexpensive and reliable method and apparatus for transmitting microbe-destroying UV light from a light source to the target of the light, with which UV light the destruction of microbes to be performed does not leave residues in/on the target to be treated and does not alter the properties of the target to be treated. Another aim is to provide a versatile, scalable and precise method and apparatus with which the destruction of microbes can, if necessary, be performed accurately targeted on a certain location and on specific microbes. A further aim is to provide a method and apparatus in which microbe-destroying UV light can easily and precisely be transmitted from a light source to targets even in awkward locations thanks to flexible fused silica fiber, and which apparatus is lightweight and easily movable from one place to another as well as being easy and quick to install into working condition. Yet another aim of the invention is to provide a solution in which, when destroying microbes, the object to be treated is illuminated and photographed with a camera via the same optical fiber element through which the microbe-destroying UV light is also transmitted. The method according to the invention is characterized by what is disclosed in the characterization part of claim 1. Correspondingly, the apparatus according to the invention is characterized by what is disclosed in the characterization part of claim 11. Other embodiments of the invention are characterized by what is disclosed in the other claims.

For realizing the aim of the invention, the invention comprises a method for transmitting microbe-destroying UV light, produced with one or more UV LEDs, from a light source to the target of the light, in which method the properties of the UV light being produced are adjusted according to the need of the target, and in which method the microbe-destroying UV light is transmitted from the light source to the target of the light via a flexible optical fiber element. Preferably, in addition to transmitting UV light that destroys microbes, also other functions enabled by optical fibers are transmitted via the same optical fiber element.

Also for realizing the aim of the invention, the invention comprises an apparatus for transmitting microbe-destroying UV light produced with one or more UV LEDs from a light source to the target of the light, which apparatus comprises a control unit for producing microbe-destroying UV light and for adjusting the properties of the UV light produced according to the need of the target, and to which control unit a flexible optical fiber element, comprising one or more optic fibers transmitting microbe-destroying UV light, is connected for transmitting the UV light produced with the apparatus from the light source to the target of the UV light. Preferably the optical fiber element comprises optical fibers intended also for other functions, in addition to the optical fibers transmitting microbe-destroying UV light.

One advantage of the method and apparatus according to the invention, more briefly described in other words as the solution according to the invention, is the lightness and small size of the apparatus, as well as its excellent precision, targetability and adjustability in destroying microbes due to, inter alia, a flexible optical fiber element, in which case the destruction of microbes can, if necessary, be performed on a precisely defined site and on specific microbes while, if so desired, leaving useful microbes that would normally die in disinfection processes currently known in the art. The use of a camera and illumination of the target via the same optical fiber element as the optical fiber element via which microbedestroying UV light is transmitted to the microbe-destroying site further improves targetability.

Another advantage is also good energy efficiency and ecofriendliness, as well as the fact that the solution according to the invention does not alter the target or the target material, nor does it leave any residues. One important advantage is also the easy scalability of the apparatus according to the treatment targets. Yet another advantage is the long service life of the UV LEDs used in the solution, in which case the servicing intervals for the apparatus are also long.

In the following, the invention will be described in more detail by the aid of some examples of its embodiment with reference to the simplified and diagrammatic drawings attached, wherein

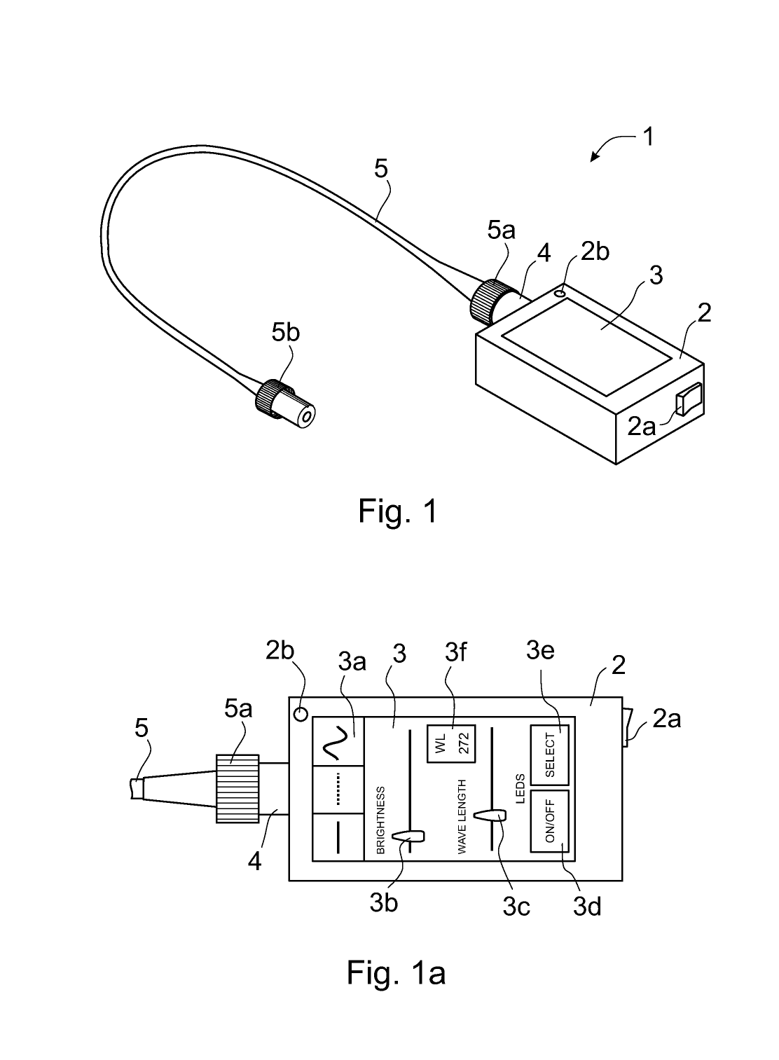

FIG. 1 presents a simplified oblique view from the side and from above of one apparatus according to the invention to be used in the method according to the invention, the apparatus comprising at least a control unit and a flexible optical fiber element,

FIG. 1a presents a simplified top view of one display panel of the control unit presented in FIG. 1,

FIG. 2 presents a front view of a LED unit, with its UV LEDs, to be used in the apparatus according to the invention, the LED unit being in its mounting base,

FIG. 3 presents a simplified and diagrammatic side view of the sectioned LED unit according to FIG. 2, with its UV LEDs and in its mounting base, wherein the UV LEDs are connected to fused silica fiber in a first manner,

FIG. 4 presents a simplified and diagrammatic side view of the sectioned LED unit according to FIG. 2, with its UV LEDs and in its mounting base, wherein the UV LEDs are connected to fused silica fiber in a second manner,

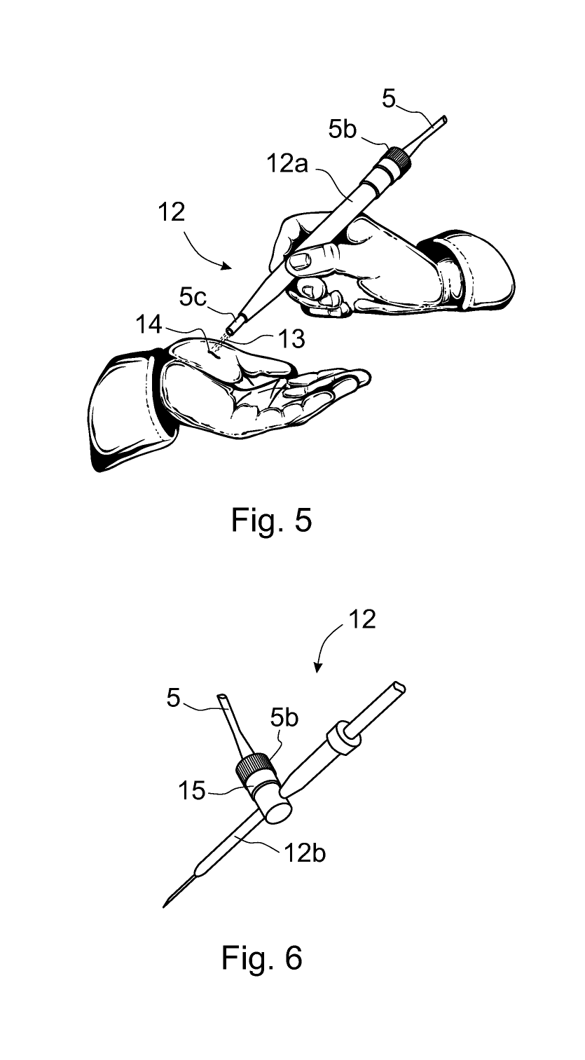

FIG. 5 presents a simplified side view of one operating device to be used in the solution according to the invention, the device being e.g. the treatment head to be used in treating wounds, disposed at the second end of the flexible optical fiber element,

FIG. 6 presents a simplified view from the side and from the top of a second operating device to be used in the solution according to the invention when connected to the optical fiber element, the device being e.g. an infusion cannula, with adapter, disposed at the second end of the flexible optical fiber element,

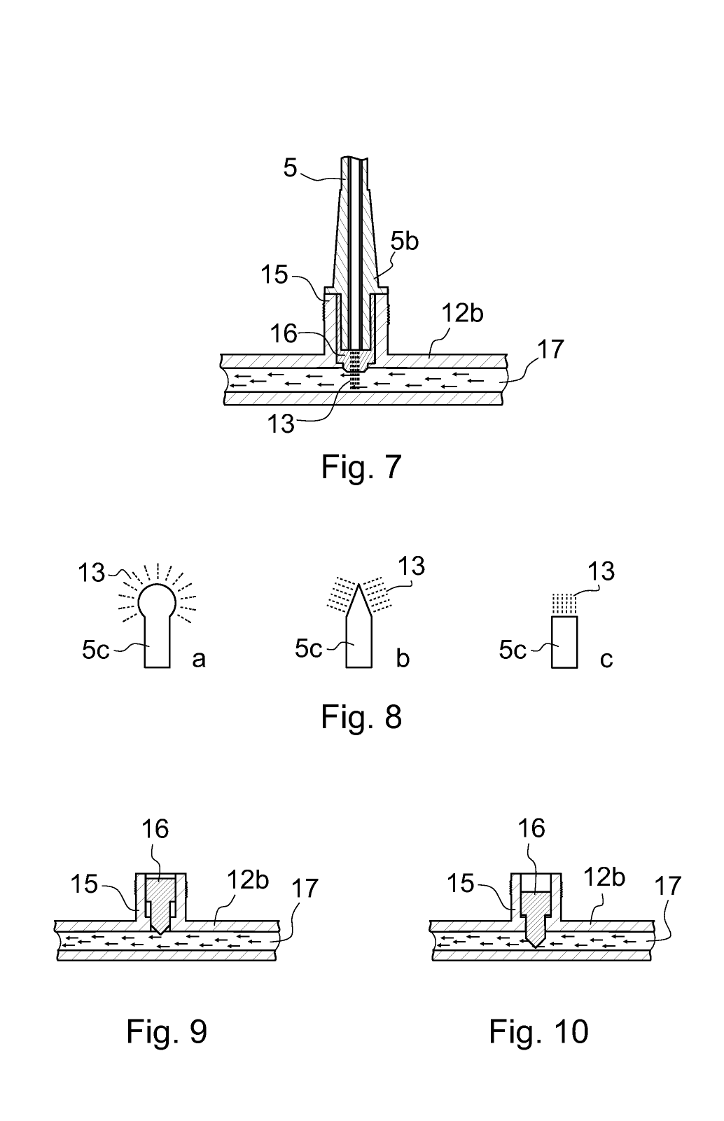

FIG. 7 presents a simplified, diagrammatic and sectioned side view of one solution according to the invention for connecting the optical fiber element to an infusion cannula,

FIG. 8 presents a simplified and diagrammatic side view of three different disinfecting or treatment heads, intended for the destruction of microbes, to be used in the method and apparatus according to the invention,

FIG. 9 presents a simplified, diagrammatic and sectioned side view of another solution according to the invention for connecting the optical fiber element to an infusion cannula, or to another disinfecting fluid, in a situation in which the fluid flow is only slightly restricted,

FIG. 10 presents a simplified, diagrammatic and sectioned side view of another solution according to the invention for connecting the optical fiber element to an infusion cannula, or to another disinfecting fluid, in a situation in which the fluid flow is significantly restricted,

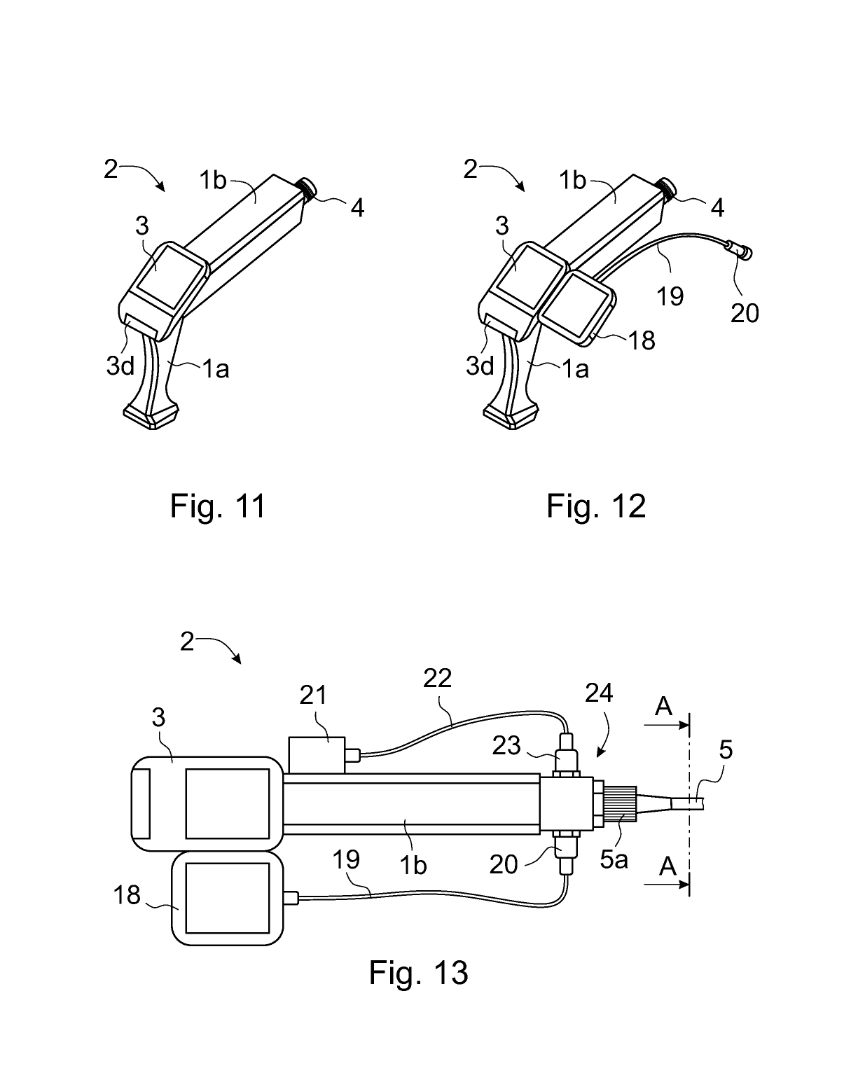

FIG. 11 presents a simplified oblique view from the side and from above of another control unit of an apparatus according to the invention to be used in the method according to the invention,

FIG. 12 presents a simplified oblique view from the side and from above of the control unit according to FIG. 11, to which control unit a camera is connected,

FIG. 13 presents a simplified top view of the control unit according to FIG. 11, to which are connected both a camera and, for the camera, a light source for the target to be disinfected,

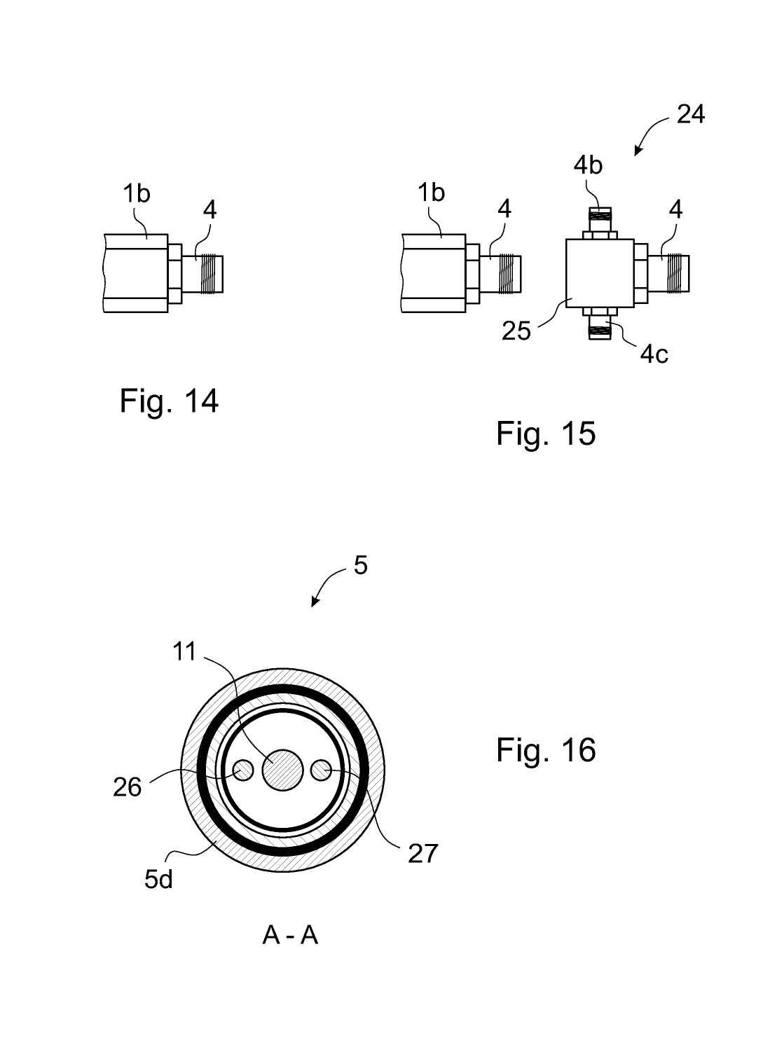

FIG. 14 presents a top view of an output connector of a control unit according to the invention, to which connector either a disinfecting head or treatment head can be connected directly, or to which the first end of an optical fiber element for transmitting UV light to a target farther away can be connected, or various adaptors can be connected,

FIG. 15 presents a top view of the output connector of the control unit according to FIG. 14 and an adapter to be connected to it, the adapter enabling the connection of additional devices, e.g. an extra light source and/or a camera, to the flexible optical fiber element, and

FIG. 16 presents a cross-section of one optical fiber element to be used in the solution according to the invention in the section A-A of FIG. 13.

FIG. 1 presents a simplified oblique view from the side and from above of one microbe-destroying, disinfecting and treatment apparatus 1 according to the invention to be used in the method according to the invention. The apparatus is small and lightweight, in which case it is portable in terms of size and weight and comprises a box-shaped, or other suitably shaped, control unit 2 and also an optical fiber element 5 to be connected to the control unit 2, inside which optical fiber element are one or more conventional fibers and/or fused silica fibers, and which optical fiber element 5 is preferably flexible. The control unit 2 preferably has means for connecting the control unit 2 to a separate stand or support.

The control unit 2 preferably comprises, disposed in an enclosure: a power source, control electronics, a UV LED unit with UV LEDs, an output connector 4 for the UV light produced, an operating switch 3d for illuminating and extinguishing the UV LEDs, a selector switch 3e for selecting the UV LEDs to be taken into use on each occasion, and also preferably adjustment switches 3a, 3b, 3c on the touch-sensitive display of the display panel 3 for adjusting the UV LEDs selected. In addition, the control unit 2 comprises a power switch 2a for switching the control unit 2 on and off, and also a signal lamp 2b that indicates when the UV light is on, and also a display screen 3f for showing the UV LEDs to be selected and the adjustment values to be adjusted with various regulators.

Correspondingly, the optical fiber element 5 to be connected to the control unit 2 comprises a one-piece or multi-piece protective cover 5d and inside the protective cover one or more fused silica optic fibers, i.e. fused silica fibers. In the same optical fiber element 5 there are, preferably inside the same protective cover 5d, also other optical fibers, e.g. for photographing and illuminating the disinfection target and/or treatment target. These optical fibers can, in fact, also be fused silica fibers. Additionally, the optical fiber element 5 comprises a first end 5a, on which are connection means for connecting the optical fiber element 5 to the output connector 4 of the control unit 2, or to an adapter therein, and a second end 5b, on which are connection means for connecting the optical fiber element 5 to the disinfecting or treatment head to be used on the disinfection target or treatment target. Preferably, the second end 5b of the optical fiber element 5 can also itself contain a disinfecting head or treatment head to be used in destroying microbes in the target. The output connector 4 functions as an intermediate piece, i.e. as an adapter, between the first end 5a of the optical fiber element 5 and the control unit 2.

FIG. 1a presents a simplified top view of one display panel 3, which is preferably e.g. a touch-sensitive display, of a control unit 2 according to the invention. Preferably the display panel 3a has the aforementioned adjustment switches 3a, 3b, 3c for adjusting the UV LEDs. The pulse character of the UV radiation is adjusted with press-type adjustment switches 3a. The first press-type adjustment switch 3a is intended for switching on UV radiation that is unbroken and remains the same in intensity, the second press-type adjustment switch 3a is intended for switching on UV radiation that is interrupted, i.e. pulse-type, but remains the same in intensity, and the third press-type adjustment switch 3a is intended for switching on UV radiation that is periodically increasing and decreasing in intensity. The third adjustment switch 3a affects the intensity of the UV radiation in such a way that the intensity changes periodically to be become lower and higher by a predefined amount on both sides of the base value of the intensity adjusted with the adjustment switch 3b. The fluctuation range of the adjustment is preferably e.g. approx. ±5% of its base value.

In addition, a first slide switch 3b on the display panel 3 is intended for adjusting the brightness of the UV light, and a second slide switch 3c is intended for adjusting the wavelength/frequency of the UV light. Adjustment of the brightness of the UV light simultaneously affects also the intensity of the UV radiation. The display panel 3 preferably also comprises a time switch, with which the radiation time can be adjusted. The properties of the UV light directed at the target are adjusted with the aforementioned adjustment switches 3a, 3b, 3c and time switch of the display panel 3, preferably manually, in such a way that the target receives a dose of UV radiation of exactly the correct properties for achieving the best possible disinfection result or treatment result. The display panel 3 preferably also has an operating switch 3d based on a touch-sensitive display, with which switch the UV LEDs of the control unit 2 are switched on and off, as well as a selector switch 3e for selecting the UV LEDs to be used. With the selector switch 3e, one or more UV LEDs are elected for use. The selection is made e.g. on the basis of e.g. the name, number, value or other individual marking of the UV LED. The one or more UV LEDs selected with the selector switch 3e are adjusted with the adjustment switches 3a-3c.

The display screen 3f is arranged to facilitate each selection event and adjustment event by presenting, which UV LED is being selected or which adjustment value is being given to the UV LEDs.

FIGS. 2-4 present a simplified view from the front and side of one LED unit 7, with UV LEDs 8 and in its mounting base 6, to be used in the solution according to the invention, the unit being inside the enclosure of the control unit 2.

In FIG. 2 the mounting base 6 and the LED unit 7 are presented detached and as viewed from the front. The mounting base 6 is preferably e.g. a metal ring, such as an aluminium ring, but it can just as well be other than ring-shaped and it can also be of a material other than metal. It can, in this case, be e.g. of rectangular shape and/or the material can be plastic or some suitable composite. Likewise, the circular plate-like LED unit 7 presented can also be of some other shape, such as e.g. rectangular. Also, the number of UV LEDs 8 in the LED unit 7 can vary. There can be e.g. only one, or any suitable number whatsoever, e.g. one of the following: 2, 3, 4, 5, 6, 7, 8, 9, 10, 11, 12, 13, 14, 15, 16, 17, 18, 19, 20, or even more.

Preferably all the UV LEDs 8 in the LED unit 7 are different to each other, at least in relation to the fundamental wavelength of the UV light they transmit, but they can also be different in terms of their threshold voltages. The fundamental wavelength/frequency is adjustable within predefined limits, preferably e.g. approx. ±5% of its base value. The adjustments are made by adjusting the voltage of the UV LEDs and/or adjusting the current going to them. Likewise, the other adjustments of the UV LEDs 8, such as adjustments of brightness/power/intensity, are made by adjusting the voltage of the UV LEDs and/or adjusting the current going to them.

The UV LEDs 8 to be used in the solution according to the invention are preferably fused silica crystal LEDs, the wavelength of which in the ultraviolet range is selected to be suitable for each purpose. A rough adjustment of the wavelength range and the power of the LEDs can be made quickly by replacing the LED unit 7 with a second LED unit 7 that has UV LEDs with different values. The mounting base 6 is fastened inside the enclosure of the control unit 2.

FIG. 3 presents a simplified and diagrammatic side view of the sectioned LED unit 7 according to FIG. 2, with its UV LEDs 8 and in its mounting base 6. In this solution the UV LEDs 8 of the LED unit 7 are connected to one fused silica fiber 11 inside the optical fiber element 5 via an output connector 4 in such a way that the internal fused silica fiber 9 of the control unit 2 connected to each UV LED 8 is adapted, e.g. by beveling the ends of the fused silica fibers 9 into one centralized fused silica fiber inside the output connector 4 or before the output connector 4, in such a way that at the output end of the output connector 4 is only one outgoing fused silica fiber, or preferably fused silica element 10, which is situated centrally in relation to the fused silica fiber 11 inside the optical fiber element 5 so that the UV light being transmitted through the fused silica element 10 hits the fused silica fiber 11 inside the optical fiber element 5 centrally.

On the outer rim of the output connector 4 is a thread, onto which the fastening element of the first end 5a of the optical fiber element 5 is screwable. One fused silica fiber 11 inside the envelope-type protective cover of the flexible part of the optical fiber element 5 is situated, by means of its adapter 11a, centrally inside the protective cover of the optical fiber element in such a way that when fastening the first end 5a of the optical fiber element 5 onto the output connector 4 of the control unit 2, the fused silica fiber 11 inside the optical fiber element 5 and the fused silica element 10 leaving from inside the output connector 4 meet each other perfectly and precisely end-to-end.

FIG. 4 presents a simplified and diagrammatic side view of the sectioned LED unit 7 according to FIG. 2, with its UV LEDs 8 and in its mounting base 6, in which solution the UV LEDs 8 are connected to the optical fiber element 5 in another manner than in the solution presented by FIG. 3. In this solution, the three UV LEDs 8 of the LED unit 7 are connected to three fused silica fibers 11 inside the protective cover of the optical fiber element 5 via the output connector 4 in such a way that one internal fused silica fiber 9 of the control unit 2 is conducted from each UV LED 8 to the output connector 4 in such a way that as many unbeveled fused silica fibers 9 lead to the first end of the fused silica element 10 inside the output connector 4 as there are UV LEDs 8 in the UV LED unit 7. The ends of the fused silica fibers 9 are arranged to hit the first end of the fused silica element 10. Correspondingly, the ends of the fused silica fibers 11 inside the optical fiber element 5 are arranged to hit the second end of the fused silica element 10 perfectly and precisely end-to-end in such a way that the UV light from each end of the fused silica fiber 9 inside the control unit 2 is transmitted directly to the end of one fused silica fiber 11 inside the optical fiber element 5.

In this solution, on the outer rim of the output connector 4 is a thread, onto which the fastening element of the first end 5a of the optical fiber element 5 is screwable. The fused silica fibers 11 inside the protective cover of the flexible part of the optical fiber element 5 are situated, by means of their adapter 11a, in the optical fiber element 5 in such a way that when fastening the first end 5a of the optical fiber element 5 onto the output connector 4 of the control unit 2, the locations of the ends of the fused silica fibers 11 inside the optical fiber element 5 and the locations of the ends of the fused silica fibers 9 inside the output connector 4 meet each other via the fused silica element 10 perfectly and precisely end-to-end. In this solution, there are just as many fused silica fibers 11 inside the optical fiber element 5 as there are UV LEDs 8 in the LED unit 7. If, for example, there are 12 units of UV LEDs 8, in this solution there are also then 12 units of fused silica fibers 11 in the optical fiber element 5.

Preferably, the fused silica element 10 can be similar in all the different embodiments because the UV light is transmitted directly through the fused silica element 10 without any scattering. In this case, the fused silica element 10 can be larger in diameter than e.g. only one fused silica fiber 9 inside the control unit 2. At its largest, the cross-sectional area of the fused silica element 10 can be e.g. always such that the light of the fused silica fibers 9 of all the UV LEDs 8 of even the largest LED element 7 can be transmitted through the fused silica element 10. The cross-sectional area of the fused silica element 10 can also vary according to the number of UV LEDs 8, or the fused silica element 10 can be completely omitted, in which case the ends of the internal fused silica fibers 9 inside the control unit 2 extend directly to the ends of the fused silica fibers 11 of the of the optical fiber element 5.

FIG. 5 presents a simplified side view of one operating device according to the invention at the second end 5b of the optical fiber element 5, the device being a treatment part to be used e.g. in microbe-destroying disinfection and treatment, which device comprises a gripping part 12a that is thicker than the flexible part of the optical fiber element 5 and at the tip of which is the actual treatment head 5c through which the UV light 13 produced in the control unit 2 by a UV LED 8, or by UV LEDs 8, is transmitted to the wound 14 to be disinfected and/or to be treated. The gripping part 12a enables a firm and supportive grip, in which case targeting the UV light at the treatment target can be done very accurately. In this solution, the gripping part 12a is connected to the general-purpose, standard-type second end of the optical fiber element 5 by means of an adapter, i.e. a fitting component. The same optical fiber elements 5 used in other usage applications can be therefore be used in this case. Preferably, the gripping end 12a can also be integrated directly into the second end of the optical fiber element 5. In such a case, when implemented in this way, the whole optical fiber element 5 is usable only for the treatment of wounds or for some corresponding use.

FIGS. 6 and 7 present the connecting of the optical fiber element 5 to another operating device 12, i.e. to an infusion cannula 12b, by means of which e.g. medication in liquid form, saline solution, nutrient fluid or blood plasma can be administered into a blood vessel.

FIG. 6 presents a simplified view, as seen obliquely from the side and top, of the second end 5b of the optical fiber element 5 when connected to an infusion cannula 12b, and FIG. 7 presents a simplified, diagrammatic and sectioned side view of one solution according to the invention for connecting the optical fiber element 5 to an infusion cannula 12b.

In the solution according to the invention, the second end 5b of the optical fiber element 5 is connected to the injection valve or injection port 15 of the infusion cannula 12b via an intermediate piece according to the purpose, i.e. via an adapter, which adapter is adapted to be placed into the injection valve 15. Preferably, the adapter comprises a fused silica element 16 suitably shaped for the injection valve 15 and having on its first end a cylindrical recess into which the second end 5b, plus its fused silica fibers 11, of the optical fiber element 5 is suitably placeable.

Correspondingly, the second end of the fused silica element 16 is arranged into contact with the fluid 17 in the infusion cannula 12b. In this case, the UV light 13 produced in the UV LED unit 7 in the control unit 2 is transmitted through the fused silica element 16 in the injection valve 15 of the infusion cannula 12b into the fluid 17 in the infusion cannula 12. The shape of the fused silica element 16 is such that it fits tightly inside the injection valve 15 and at its second end meets the fluid 17 in the infusion cannula 12b in such a way that the fluid 17 does not at any stage come into contact with the second end 5b of the optical fiber element 5. In this case, the optical fiber element 5 does not need to be discarded after use, but instead it is sufficient to discard the cheap fused silica element 16 functioning as an adapter.

FIG. 8 presents a simplified and diagrammatic side view of three different treatment heads 5c to be used in the method and apparatus according to the invention. FIG. 8a presents an omnidirectional ball-shaped treatment head 5c, FIG. 8b presents a treatment head 5c intended for e.g. cancer treatment, and FIG. 8c presents a treatment head 5c intended for precise nursing, e.g. for treating wounds.

Preferably there are many different treatment heads 5c and/or disinfecting heads, always best suited for the specific use at the time and provided with appropriate ancillaries. One advantageous solution is the disinfection of fluid materials to be dispensed into sales and storage receptacles with disinfection heads suited for the purpose. For example, when dispensing tomato sauce, or some other corresponding fluid material, into a bottle, it is advantageous to dispose a disinfection head of the apparatus according to the invention between the dispensing device and the bottle in connection with the material flow. When the disinfection properties of the UV light are adjusted with the control unit 2 to be suitable, the material dispensed into the bottle is simultaneously disinfected in the dispensing phase.

Preferably, the second end of the fused silica element 16 is shaped to function as a valve and the location of the second end of the fused silica element 16 in relation to the fluid 17 in the infusion cannula 12b is arranged to be adjustable. In such a case, the speed and/or flow rate of the fluid in the infusion cannula 12b can be adjusted by means of the fused silica element 16, in which case by means of this adjustment and the adjustments of the control unit 2 it is possible to achieve an extremely precise disinfection effect on the fluid 17 in the infusion cannula 12b. FIGS. 9 and 10 present this type of solution, in which the second end, i.e. the end touching the fluid 17 in the infusion cannula 12b, of the fused silica element 16 is shaped to be essentially conical. FIG. 9 presents a situation in which the adjustment is almost at its maximum, in which case the fused silica element 16 is almost in its open position. In this case, the liquid 17 in the infusion cannula 12b flows almost unrestrictedly. Correspondingly, FIG. 10 presents a situation in which the fused silica element 16 is almost in its closed position, in which case the fused silica element 16 shuts off the fluid flow almost completely. For the sake of clarity, the arrangement for moving and adjusting the fused silica element 16 is not presented in FIGS. 9 and 10.

FIG. 11 presents a simplified oblique view from the side and from above of another control unit 2 of an apparatus 1 according to the invention to be used in the method according to the invention. Instead of a box-shaped control unit 2, in this solution the control unit 2 is essentially pistolshaped. The control unit 2 comprises a handle 1a and a pointer part 1b, at the free end of which is an output connector 4 for fastening the first end 5a of the optical fiber element 5 to the control unit 2. The output connector 4 is presented in more detail in FIGS. 14 and 15. Additionally, the control unit 2 according to FIG. 11 has an essentially similar display panel 3, with its functions and operating switches 3a-3f and also power switch, as in the control unit 2 according to FIGS. 1 and 1b, as well as also essentially similar control electronics and UV LED unit 7 with UV LEDs 8.

Also in the control unit 2, preferably in its handle 1a, are batteries or accumulators functioning as a power source, which keep the center of mass of the control unit 2 as low as possible, thereby ensuring that the control unit 2 remains upright when being used. If necessary, a lightweight additional support is fastenable to the control unit 2, preferably to its pointer part 1b, which support contributes to keeping the control unit 2 upright.

FIGS. 12 and 13 present the connecting to the control unit 2 of separate auxiliary devices and additional devices belonging to the solution according to the invention. In the solution according to FIG. 12, connected to the control unit 2 is a camera 18 for photographing the target to be disinfected and/or to be treated, and/or for guiding the second end 5b of the optical fiber element 5 to the target to be disinfected and/or to be treated. In the solution according to FIG. 13, also connected to the control unit 2 is a light source 21 for illuminating for a camera 18 the target to be disinfected and/or to be treated. The camera 18 is provided with an optical fiber 19 for connecting the camera 18, via a connector 20 and adapter 24, to the optical fiber element 5, and, correspondingly, the light source 21 is provided with an optical fiber 22 for connecting the light source 21, via a connector and adapter 24, to the optical fiber element 5. In this way, a real-time video image and/or still image can be taken with the camera 18 via the optical fiber element 5 primarily intended for transmitting UV light and at the same time also the view of the camera 18 can be illuminated with the light source 21 via the optical fiber element 5. In addition, the camera 18 has means for recording and/or transferring to a separate data means, such as a tablet, computer or separate memory, the video and/or still image materials taken with the camera 18. Image material is arranged to be transferred either wirelessly, e.g. with a WiFi connection, or by wireline or directly from the camera to a memory stick.

FIG. 14 presents a top view of an output connector 4 of a control unit 2 according to the invention, to which connector either a microbe-destroying disinfecting head or microbedestroying treatment head can be connected directly, or the first end 5a of an optical fiber element 5 for transmitting UV light to a target farther away can be connected. Various adapters can also be connected to the output connector 4 for connecting different auxiliary and additional devices, e.g. an extra light source 21 and a camera 18, to the flexible optical fiber element 5. One preferred adapter 24 to be connected to the output connector 4 is presented in FIG. 15. The adapter 24 has a frame part 25 and connection means for fastening the adapter to the output end 4 of the control unit 2 in such a way that the UV light transmitted by the control unit 2 travels essentially unchanged through the adapter 24 to the output end 4 of the adapter, which is essentially similar to the output end 4 of the control unit 2. Additionally, the adapter 24 has a first additional connector 4a for connecting an optical fiber 22 of the light source 21 via the adapter 24 to the optical fiber element 5, and a second additional connector 4b for connecting an optical fiber 19 of the camera 18 via the adapter 24 to the optical fiber element 5. The adapter 24 can also have other additional connectors. The camera 18 and light source 21 can be connected to the optical fiber element 5 also in some other manner than by means of the adapter 24 presented above. In such a case, e.g. the additional connectors 4a and 4b can be e.g. directly in the control unit 2.

FIG. 16 presents a cross-section of one optical fiber element 5 to be used in the solution according to the invention, in the section A-A presented by FIG. 13. In the solution according to the invention, all the optical fibers 11, 26, 27 to be used for different functions are situated inside the same envelope-type protective cover 5d, which protective cover 5d preferably comprises a number of different protective and support layers. There is preferably only one fused silica fiber 11 transmitting UV light in the optical fiber element 5, but as already stated there can also be more of them. Likewise, there are one or more optical fibers 26 for the light source 21 in the optical fiber element 5 and also one or more optical fibers 27 for the camera 18 in the optical fiber element 5. The optical fibers 26 and 27 are preferably also fused silica fibers, but they can be of some other material. What is essential is that the optical fibers 26 and 27 of the light source 21 and of the camera 18 are in the same protective cover 5d as the fused silica fibers 11 transmitting UV light and that they extend inside the protective cover 5d from the first end 5a to the second end 5b of the optical fiber element 5 in essentially the same manner as the fused silica fibers 11 intended for UV light.

Presented above are the various operating devices 12 to be connected to the second end 5b of the optical fiber element 5. As already stated earlier in this description, the second end 5b can preferably itself function as an operating device 12, in which case a separate operating device 12 and the intermediate piece fitted to it, i.e. the adaptor, are not needed.

With the method according to the invention, the UV LED light is transferred, i.e. transmitted, with a flexible fiber element that conducts UV light very well, such as with an optical fiber element 5 comprising one or more fused silica fibers 11, from the UV light source, i.e. from the UV LEDs 8 of the control unit 2, to the target. One or more of the following adjustment items, one of which adjustments can simultaneously affect another, is adjusted with the control unit 2, if necessary, in conjunction with the transfer of the UV light, either before the transfer or during the transfer: the intensity, brightness, wavelength, frequency, duration of action and/or pulse character, i.e. whether the light is continuous or pulsed, of the UV light. Another adjustment that can be mentioned is the selection of the LED or LEDs.

The radiant power/intensity of the UV light is adjusted e.g. by adjusting the brightness of the UV light. All the adjustments can be made in conjunction with the same microbedestruction session, disinfection session or treatment session either to one aforementioned adjustment items, but if necessary also to more than one adjustment item. Thus, for example, only the intensity can be adjusted, but if necessary also other adjustment items, e.g. the wavelength and/or frequency of the UV light can be adjusted in conjunction with the same microbedestruction session, disinfection session or treatment session. All the adjustments of the UV LEDs 8, such as adjustments of brightness/power/intensity and wavelength/frequency, are made by adjusting the voltage of the UV LEDs 8 and/or adjusting the current going to them.

In the method according to the invention, a flexible fiber element that conducts UV light very well, such as an optical fiber element 5, is connected between one or more UV LEDs 8 of the UV light source, i.e. of the control unit 2, and the microbedestruction target, disinfection target or treatment target, i.e. operating target, in such a way that the first end 5a of the optical fiber element 5 is fastened to the control unit 2 via a suitable intermediate piece, such as via the output connector 4 of the control unit 2 or an adapter 24, and the second end 5b of the optical fiber element is fastened via a suitable intermediate piece, i.e. via an adapter, to an operating device 12, such as e.g. to a gripping part 12a provided with a wound treatment head 5c, or to an injection valve of an infusion cannula 12b, or to some other necessary operating device 12. Preferably, especially in short fiber elements, an operating device 12 can be integrated in the secand end of the optical fiber element 5 without a separate intermediate piece. In such a case, the optical fiber element 5 in question is used only for limited targets, e.g. only for the disinfection and treatment of wounds.

Preferably the location and movement of the second end 5b of the optical fiber element is monitored via a camera 18, with which real-time video images and/or a still image is taken via the optical fiber element 5 of the proximity of the second end 5b of the optical fiber element and of the target to be disinfected and/or to be treated. Likewise, the field of vision of the camera 18 is illuminated preferably by means of a light source 21 via the optical fiber element 5.

The different solutions and features presented above can be inventive features together with one or more other features of the invention.

It is obvious to the person skilled in the art that the invention is not limited solely to the examples described above, but that it may be varied within the scope of the claims presented below. Thus, for example, the structure and operation of the control unit, the optical fiber element and the operating devices to be used in disinfection targets or treatment targets can also be different to what is presented above.

1.-21. (canceled)

22. A method for transmitting microbe-destroying UV light produced by one or more UV LEDs from a light source in a control unit to the target of the UV light, in which method the properties of the UV light being produced are adjusted according to the need of the target, and in which method the UV light is transmitted from the light source to the target of the light via a flexible optical fiber element comprising a first end and a second end, and in which method other functions, such as photographing and/or illuminating the targets, enabled by optical fibers are transmitted via the same optical fiber element, wherein each UV LED is mounted on a replaceable LED unit and the microbe-destroying UV light produced by each UV LED is roughly adjusted by replacing the current LED unit by another LED unit that has UV LEDs with different values, and that the microbe-destroying UV light produced by each UV LED is transmitted from the control unit via an intermediate piece to the first end of the flexible optical fiber element, and the microbe-destroying UV light transmitted through the optical fiber element is transmitted to an operating device on the second end of the optical fiber element via an intermediate piece.

23. The method according to claim 22, wherein microbe-destroying UV light is produced in the portable control unit and the first end of a flexible optical fiber element is fitted in front of a light source in the control unit to receive the UV light produced by each UV LED of the light source and the second end of the flexible optical fiber element is fitted to convey to a microbe-destroying treatment point the UV light produced by each UV LED and transmitted through the flexible optical fiber element for destroying microbes in/on the point being treated.

24. The method according to claim 22, wherein at the target the microbedestroying UV light transmitted through the flexible optical fiber element is transmitted to the target via the operating device.

25. The method according to claim 22, wherein with the control unit the intensity and/or wavelength and/or frequency and/or pulse character and/or duration of action of the microbe-destroying UV light to be transmitted to the target is adjusted before transmission of the UV light to the target and/or during transmission of the UV light.

26. The method according to claim 25, wherein the intensity and/or wavelength and/or frequency of the microbe-destroying UV light to be transmitted to the target is adjusted by adjusting the voltage of each UV LED and/or the current going to it.

27. The method according to claim 22, wherein the microbe-destroying UV light to be transmitted to the target is produced by means of one or more fused silica crystal LEDs, and the UV light produced in this way is transmitted from the control unit via one or more fused silica fibers situated inside the optical fiber element to the target.

28. The method according to claim 22, wherein a camera is connected to the control unit for photographing the target for microbe destruction, and/or for guiding the second end of the optical fiber element to the target for microbe destruction.

29. The method according to claim 28, wherein a light source is connected to the control unit for illuminating the target for microbe destruction for the camera.

30. The method according to claim 29, wherein a camera and a light source are connected with their own optical fibers to the optical fiber element primarily intended for transmitting microbe-destroying UV light, for taking a real-time video image and/or still images, via the optical fiber element, of the target for microbe destruction and/or for guiding the second end of the optical fiber element to the target for microbe destruction.

31. An apparatus for transmitting microbe-destroying UV light produced with one or more UV LEDs from a light source to the target of the light, which apparatus comprises a control unit for producing microbe-destroying UV light and for adjusting the properties of the UV light produced according to the need of the target, and to which control unit a flexible optical fiber element, comprising a first end and a second end and one or more optic fibers transmitting microbe-destroying light, is connected for transmitting the UV light produced with the apparatus from the light source to the target of the microbe-destroying UV light, which optical fiber element further comprises optical fibers intended for other functions, wherein the apparatus comprises a replaceable LED unit on which the UV LEDs are mounted, and the microbedestroying UV light produced by each UV LED is arranged to be roughly adjusted by replacing the current LED unit by another LED unit that has UV LEDs with different values, and that the apparatus comprises a first intermediate piece for transmitting the microbe-destroying UV light produced by each UV LED from the control unit to the first end of the flexible optical fiber element, and a second intermediate piece for transmitting the microbe-destroying UV light transmitted through the optical fiber element to an operating device on the second end of the optical fiber element.

32. The apparatus according to claim 31, wherein the control unit where the microbe-destroying UV light is arranged to be produced is a portable control unit, and the first end of a flexible optical fiber element is fitted in front of a light source in the control unit to receive the microbe-destroying UV light produced by each UV LED of the light source, and the second end of the flexible optical fiber element is adapted to convey to a treatment point to be destroyed of microbes the microbe-destroying UV light produced by each UV LED and transmitted through the flexible optical fiber element.

33. The apparatus according to claim 31, wherein at the target the microbedestroying UV light transmitted through the flexible optical fiber element is arranged to be transmitted to the target via an operating device.

34. The apparatus according to claim 31, wherein in the control unit are adjusting means for adjusting the intensity and/or wavelength and/or frequency and/or pulse character and/or duration of action of the microbe-destroying UV light to be transmitted to the target before transmission of the UV light to the target and/or during transmission of the UV light.

35. The apparatus according to claim 31, wherein the apparatus comprises one or more fused silica crystal LEDs for producing microbe-destroying UV light to be transmitted to a target, and one or more fused silica fibers situated inside the flexible optical fiber element for transmitting the UV light produced in the apparatus from the control unit to the target.

36. The apparatus according to claim 31, wherein a camera is connected to the control unit for photographing the target for microbe destruction, and/or for guiding the second end of the optical fiber element to the target for microbe destruction.

37. The apparatus according to claim 36, wherein a light source is connected to the control unit for illuminating the target for microbe destruction for the camera.

38. The apparatus according to claim 37, wherein a camera and a light source are connected with their own optical fibers to the optical fiber element primarily intended for transmitting microbe-destroying UV light, for taking a real-time video image and/or still images via the optical fiber element of the target for microbe destruction and/or for guiding the second end of the optical fiber element to the target for microbe destruction.

39. The apparatus according to claim 38, wherein, in the optical fiber element, in addition to one or more optical fibers intended for transmitting the UV light used in the destruction of microbes, there is at least one optical fiber for a camera and at least one optical fiber for a light source.

40. The apparatus according to claim 39, wherein the optical fiber for the light source and the optical fiber for the camera are inside the same envelope-type protective cover in the optical fiber element as the optical fibers transmitting microbe-destroying UV light.
